(19) United States
(12) Patent Application Publication
(10) Pub. No.: US 2004/0248190 A1
(43) Pub. Date: Dec. 9, 2004

(54) Compositions and kits for differential diagnosis of hydatidiform moles and methods of using the same

(76) Inventors: Benjamin Tycko, Demarest, NJ (US); Harshwardhan Thaker, Tenafly, NJ (US); Anjana Saxena, New York, NY (US)

Correspondence Address: Brown Raysman Millstein Felder & Steiner LLP, 163 Madison Avenue, P.O. Box 1989, Morristown, NJ, 07962-1989, US

(21) Appl. No.: 10/456,778

(22) Filed: Jun. 6, 2003

(51) Int. Cl. G01N 33/53

(52) U.S. Cl. 435/7.1

(57) ABSTRACT

The present invention provides various antibody and nucleic acid compositions useful in the differential diagnosis of hydatidiform mole. In particular, the invention discloses compositions comprising antibodies specifically targeted to bind to amino acid residues 102-120 or 134-152 of the human IPL protein, as well as compositions comprising nucleic acids that specifically hybridize to target nucleic acids encoding amino acid residues 102-120 or 134-152 of the human IPL protein. The invention further discloses methods of using the subject antibody and nucleic acid compositions to differentially diagnose complete hydatidiform mole in a subject. Also provided are kits for diagnosing complete hydatidiform mole, where the kits comprise the antibodies or nucleic acids of the present invention.

### STATEMENT OF GOVERNMENT INTEREST

This invention was made with government support from the NIH Grant No. R01 CA 60765. Accordingly, the United States government may have certain rights in this invention.

### COPYRIGHT NOTICE

A portion of the disclosure of this patent document contains material subject to copyright protection. The copyright owner has no objection to the facsimile reproduction by anyone of the patent document or the patent disclosure, as it appears in the Patent and Trademark Office patent file or records, but otherwise reserves all copyright rights.

### BACKGROUND OF THE INVENTION

Gestational trophoblastic disorders (GTDs) include a number of benign and malignant growths arising from placental tissue following abnormal fertilization in a preceding pregnancy. (Kudelka, et al., Gestational Trophoblastic Tumors, in Cancer Management: A Multidisciplinary Approach. 5^{th} Ed. 2001). While GTDs include choriocarcinomas, placental site trophoblastic tumors, epitheloid trophoblastic tumors, exaggerated placental sites, and placental site nodules, by far the most common GTDs are hydatidiform moles (HM), which account for approximately 1 in 2000 gestations in the United States and up to 1 in 200 gestations in certain Asiatic countries. (Beers, M. H. and Verkow, R., Eds., Gestational Trophoblastic Disease, in the Merck Manual, 17^{th} Edition, 1999). The presenting symptoms of HM include vaginal bleeding, excessive uterine enlargement for gestational age, lack of fetal movement or heart sounds and severe hyperemesis gravidarum resulting from elevated beta-HCG levels. (Beers and Berkow, supra, 1999). Ultrasound examination yields a characteristic “snowstorm” appearance that suggests a diagnosis of hydatidiform mole.

Hydatidiform moles are generally classified on the basis of histopathology and genetic origin as either complete hydatidiform moles (CHM) or partial hydatidiform moles (PHM). (Roberts, D. G. and Mutter, G. L., Advances in the molecular biology of gestational trophoblastic disease. J. Reprod. Med. 39(3):201-8, 1994). Histologically, complete hydatidiform moles are characterized by (i) abnormal villi with large oval cystic villous inclusions, being either avascular or having collapsed empty vessels, (ii) excessive trophoblast proliferation, and (iii) marked hydropic change with prominent cisterns. While a very rare form of familial CHM has been described which is biparental, the overwhelmingly vast majority of CHM are androgenetic (i.e., paternally uniparental), having 46 chromosomes resulting either from (i) the fertilization of an enucleate ooctye by two sperm to yield the full chromosomal complement, or (ii) the fertilization of an enucleate oocyte by a single sperm, followed by a genetic doubling. (Roberts, D. G. and Mutter, G. L., Advances in the molecular biology of gestational trophoblastic disease. J. Reprod. Med. 39(3):201-8, 1994; Fisher, R. A., et al., The maternally transcribed gene p57(KIP2) (CDNK1C) is abnormally expressed in both androgenetic and biparental complete hydatidiform moles. Hum. Mol. Genet. 11:3267-72, 2002).

Partial hydatidiform moles have a different histological presentation, as samples often have vascularization, with less marked trophoblast proliferation and hydropic change than CHM. These moles generally result from a dispermic conception, yielding a triploid conceptus (69 chromosomes) having one maternal chromosomal complement and two paternal chromosomal complements. (Roberts, D. G. and Mutter, G. L., supra, 1994; Fisher, R. A., supra, 2002). A partial mole may even show some limited fetal development, although the fetus rarely survives past the 9^{th} week.

Increasingly, however, improvements in prenatal care mean that hydatidiform moles are evacuated in the very early stages of gestation, so that the differential diagnosis of complete hydatidiform mole from partial mole, or even from hydropic abortion bearing molar characteristics, becomes more difficult. Such a diagnosis, however, is crucial for issues related to patient management, since up to 20% of CHM may recur or progress to malignancy (Roberts, D. G. and Mutter, G. L., supra, 1994; Fukunaga, M. Immunohistochemical characterization of p57(KIP2) expression in early hydatidiform moles. Hum Pathol. 33:1188-92, 2002), while PHM rarely (and hydropic abortions almost never) persist or progress to carcinoma following evacuation.

One possible solution is to use immunohistochemical or immunological techniques to detect proteins that are differentially expressed in cases of complete hydatidiform mole. That certain proteins are differentially expressed in CHM is supported by the findings of Bartofi, et al. (hereinafter “Bartofi”), as reported in “Protein profiling of complete mole and normal placenta using ProteinChip analysis on laser capture microdissected cells”. (Gyn. Oncol., 88:424-428, 2003). Using surface-enhanced laser desorption/ionization mass spectroscopy technology, Bartofi found three metal binding polypeptides appearing in significantly lower levels in complete moles than in normal placental tissue. No attempt was made, however, to further characterize these unidentified polypeptides, or to determine whether the reduced expression was specifically and differentially characteristic of complete mole versus partial mole.

One attractive source for differential or diagnostic markers associated with complete hydatidiform moles is protein products of the so-called “imprinted genes”. Genomic or parental imprinting is an epigenetic phenomenon that causes parent-of-origin-dependent silencing or imprinting of alleles, leading to the monoallelic or biased allelic expression of imprinted genes in the conceptus. Based on an increasing number of examples, it has been suggested that this phenomenon may disproportionately affect loci that control pre- and post-natal growth, as well as certain aspects of neonatal and maternal behavior. (B. Tycko and I. M. Morison, Physiological functions of imprinted genes. J. Cell Physiol. 192:245-58, 2002). According to the prevailing “intergenomic conflict” theory, gene imprinting is a response to the competing maternal and paternal drives to control allocation of maternal resources to each conceptus. Id. That is, in settings of multiple paternity, the father will propagate his genome most efficiently if his germline imprints genes in a pattern that promotes the growth of his offspring, both in utero and in the post natal period. Id. The mother, by contrast, is postulated to propagate her genome more successfully by imprinting genes to prevent undue metabolic demands on her resources by any single conceptus or pregnancy. Id. Therefore imprinted genes that are paternally expressed/maternally silenced are predicted to promote growth of the offspring, or in some other way increase demands on maternal resources, while imprinted genes with the opposite direction of imprinting (i.e., maternally expressed/paternally imprinted) should have the opposite effect. Id.

Using abnormalities in the imprinting of a gene or a population of genes to diagnose a disease or determine a predisposition for a disease was suggested by A. P. Feinberg, in U.S. Pat. No. 6,235,474, entitled “Methods and Kits for Diagnosing and Determination of the Predisposition for Diseases” (hereinafter referred to “Feinberg”). (See also, U.S. patent application Ser. No. 20010007749, entitled “Methods and Kits for Diagnosing and Determination of the Predisposition for Diseases”; still further, see: Lee, M. P. and Feinberg, A. P., Genomic Imprinting of a Human Apoptosis Gene Homologue, TSSC3. Cancer Res. 58:1052-1056, 1998; and A. P. Feinberg, Imprinting of a Genomic Domain of 11p15 and Loss of Imprinting in Cancer. Cancer Res. 59(Suppl):1743s-1746s, 1999). In particular, Feinberg discloses a correlation between the presence or absence of “loss of imprinting” in a subject's somatic cells and the presence of disease or the risk of contracting a disease. According to the disclosure in Feinberg, a “loss of imprinting” is defined as the case where the particular gene that is being examined is normally imprinted, but in the disease state is abnormally not imprinted, usually due to mutation or defects in methylation. In an example supporting his disclosure, Feinberg analyzed eighty specimens derived from colorectal cancer patients for the loss of imprinting of each allele of the IGF2 gene. Using quantitative PCR assays, Feinberg determined that a frequent loss of imprinting of one allele of the IGF2 gene exists in colon cancer, even in the normal tissue of such cancer patients.

Feinberg addresses methods to diagnose a disease or cancer occurring due to a loss of imprinting. He does not address, however, any methods to diagnose disease or cancer occurring due to uniparental disomy (i.e., parthenogenesis or androgenesis), as would be the case with complete hydatidiform mole. Further, Feinberg does not disclose or suggest a correlation between any particular imprinted gene and complete hydatidiform mole.

Certain imprinted genes that influence placental growth and development in mice include: Igf2 (Baker, J., et al., Role of insulin-like growth factors in embryonic and postnatal growth. Cell. 75:73-82, 1993; Caspary, T., et al., Oppositely imprinted genes p57(Kip2) and igf2 interact in a mouse model for Beckwith-Wiedemann syndrome. Genes Dev. 13:3115-24, 1999); Igf2r (Ludwig, T., et al., Mouse mutants lacking the type 2 IGF receptor (IGF2R) are rescued from perinatal lethality in Igf2 and Igf1r null backgrounds. Dev Biol. 177:517-35, 1996); Mash2/Ascl2 (Guillemot, F., et al., Essential role of Mash-2 in extraembryonic development. Nature. 371:333-6, 1994); p57Kip2/Cdkn1c (Caspary et al., supra, 1999; Takahashi, K., et al., p57(Kip2) regulates the proper development of labyrinthine and spongiotrophoblasts. Mol. Hum. Reprod. 6:1019-25, 2000; Zhang, P., C., et al., Cooperation between the Cdk inhibitors p27(KIP1) and p57(KIP2) in the control of tissue growth and development. Genes Dev. 12:3162-7, 1998); Peg1/Mest (Lefebvre, L., et al., Abnormal maternal behaviour and growth retardation associated with loss of the imprinted gene Mest. Nat. Genet. 20:163-9, 1998); Peg3 (Li, L., et al., Regulation of maternal behavior and offspring growth by paternally expressed Peg3. Science. 284:330-3, 1999); Esx1 (Li, Y., and R. R. Behringer, Esx1 is an X-chromosome-imprinted regulator of placental development and fetal growth. Nat. Genet. 20:309-11, 1998); and Ipl (Frank, D., et al., Placental overgrowth in mice lacking the imprinted gene Ipl. Proc. Natl Acad. Sci. U.S.A. 99:7490-5, 2002).

One of these genes, the paternally imprinted p57_{KIP2}, was the target of an immunohistochemical test to differentially diagnose CHM, as is disclosed by M. Fukunaga in “Immunohistochemical characterization of p57_{KIP2} expression in early hydatidiform moles” (hereinafter, “Fukunaga”). (Hum. Pathol. 33(12):1188-1192, 2002). In Fukunaga, a monoclonal antibody against p57_{KIP2} protein was used to evaluate expression of this paternally imprinted gene in 20 diploid hydropic abortions, 20 triploid PHM and 44 diploid CHM. It was found that p57_{KIP2} expression in cytotrophoblasts and villous stromal cells was either absent (37 cases) or very low (7 cases) in the CHM, while the majority of hydropic abortions and partial moles showed p57_{KIP2} levels comparable to those observed in normal placental tissue. Staining in villous intermediate trophoblasts of the CHM, however, was also normal or near normal.

Accordingly, the use of p57_{KIP2} expression as a marker for the differential diagnosis of complete hydatidiform mole, while useful, is ambiguous, at least to the extent that expression of the marker is seen in certain placental cell types (i.e., villous intermediate trophoblasts) regardless of pathological origin. This characteristic may be due to an incomplete or relaxed imprinting of p57_{KIP2} in certain cell types. As a result, p57_{KIP2} is a specific marker for androgenetically derived mole only in certain cell types of the placenta, therefore requiring additional clinical and histological determinations before a differential diagnosis could be made.

An additional gene of interest as a potential marker for the differential diagnosis of complete hydatidiform mole is IPL (Imprinted in Placenta and Liver). First identified by Qian, et al. (hereinafter, “Qian”), in “The IPL gene on chromosome 11 p15.5 is imprinted in humans and mice and is similar to TDAG51, implicated in Fas expression and apotosis” (Hum. Mol. Genetics. 6(12):2021-2029, 1997), both human and mouse IPL genes show tissue-specific expression and functional imprinting, with the maternal allele active and the paternal allele relatively inactive. Human IPL is highly expressed in the placenta, and shows low but detectable expression in fetal and adult liver and lung. Id.

Frank, et al. (hereinafter, “Frank I”) raised antibodies against mouse IPL in order to determine expression patterns in the extraembryonic tissues of the mouse. (A novel pleckstrin homology-related gene family defined by IPL/Tssc3, TDAG51, and Tih1: tissue specific expression, chromosomal location, and parental imprinting. Mamm. Gen. 10:1150-1159). IPL protein expression was found to be restricted to the labyrinthine trophoblast of the placenta and the visceral endoderm cells of the yolk sac. Subsequently, Frank, et al. (hereinafter, “Frank II”) generated two lines of mice with germ line deletions of the Ipl gene. While the Ipl knock-out mice were viable, there was consistent overgrowth of the Ipl null placentas, with concordant expansion of the spongiotrophoblast. No corresponding fetal overgrowth was observed. Accordingly, it was determined that, at least in mice, Ipl expression is an antagonist to placental growth.

However, none of Qian, Frank I or Frank II discloses or suggests the use of Ipl as a differential diagnostic marker for complete hydatidiform mole. Nor do any of these references disclose the generation of antibodies against the human IPL protein or to any particular epitope of the human IPL protein.

Accordingly, in light of the foregoing, there exists a need for a simple and accurate diagnostic test, preferably an immunological test, to rapidly differentiate between androgenetic forms of gestational trophoblastic disease, such as complete hydatidiform mole, and biparental forms of gestational trophoblastic disease, such as partial hydatidiform mole.

### SUMMARY OF THE INVENTION

The present invention discloses a number of compositions directed to the detection of human IPL expression, and more specifically, to the detection of IPL expression in human tissue associated with complete hydatidiform mole. The Ipl (Imprinted in Placenta and Liver) gene (Qian, et al., The IPL gene on chromosome 11p15.5 is imprinted in humans and mice and is similar to TDAG51, implicated in Fas expression and apoptosis. Hum. Mol. Genet. 6(12):2021-9, 1997), also known as tumor suppressing subtransferable candidate 3 (TSSC3) (Feinberg, A. P., Imprinting of a genomic domain of 11p15 and loss of imprinting in cancer: an introduction. Cancer Res. 59(7 Supp):1743S-1746S, 1999), tumor suppressing subchromosomal transferable fragment cDNA3 (Hu, et al., A 2.5-Mb transcript map of a tumor-suppressing subchromosomal transferable fragment from 11p15.5, and isolation and sequence analysis of three novel genes. Genomics. 46(1):9-17, 1997), and p17-Beckwith-Wiedemann region 1C (Schwienbacher, et al., Transcriptional map of 170-kb region at chromosome 11p15.5: identification and mutational analysis of the BWR1A gene reveals the presence of mutations in tumor samples. Proc. Natl Acad. Sci. U.S.A. 95(7):3873-8, 1998), is expressed as a single pleckstrin homology domain with short N- and C-terminal extensions. The inventors show herein that there is an absence or near absence of Ipl transcription (i.e., IPL mRNA) or translation (i.e., IPL protein expression) in cases of androgenetically derived complete hydatidiform mole, while IPL expression (i.e., transcription and/or translation) in cases of biparentally derived placental tissues, such as normal placenta or partial hydatidiform mole, is normal or near normal. Accordingly, the inventors provide herein a valuable diagnostic marker for the differential diagnosis of complete hydatidiform mole.

Accordingly, in one aspect of the invention, a composition is provided which comprises an anti-IPL antibody or an antigen binding fragment thereof, wherein the antibody or antibody fragment specifically binds to human IPL protein. In a preferred embodiment of the invention, an antibody or antigen binding fragment of the present invention is targeted and specifically binds to amino acid residues QNRRALQDFRSRQERTAPA (SEQ ID NO.: 1), consisting of amino acid residues 102-120 of human IPL protein. In an alternate preferred embodiment, an antibody or antigen binding fragment of the present invention is targeted and specifically binds to amino acid residues PSEPSEPSRPSPQPKPRTP (SEQ ID NO.: 2), consisting of amino acid residues 134-152 of human IPL protein.

The invention further provides a composition comprising an anti-IPL nucleic acid, wherein the anti-IPL nucleic acid specifically binds to a target nucleic acid encoding the human IPL protein. In a preferred embodiment of the invention, the anti-IPL nucleic acid of the composition specifically binds or hybridizes to a target nucleic acid encoding amino acid residues QNRRALQDFRSRQERTAPA (SEQ ID NO.: 1), consisting of amino acid residues 102-120 of human IPL protein. In an alternate preferred embodiment, the present invention provides a composition comprising an anti-IPL nucleic acid, wherein the nucleic acid specifically binds or hybridizes to a target nucleic acid encoding amino acid residues PSEPSEPSRPSPQPKPRTP (SEQ ID NO.: 2), consisting of amino acid residues 134-152 of human IPL protein.

The invention still further provides a method for differentially diagnosing complete hydatidiform mole in a subject, comprising the steps of contacting a sample of suspected molar tissue from the subject with an anti-IPL antibody or an antigen binding fragment of the present invention, and thereafter detecting complexes formed between the anti-IPL antibody or antigen binding fragment and human IPL in the sample. In a preferred embodiment of the invention, the antibody or antigen binding fragment of the disclosed method specifically binds to amino acid residues 102-120 of human IPL protein. In an alternate preferred embodiment, the antibody or antigen binding fragment of the disclosed method specifically binds to amino acid residues 134-152 of human IPL protein.

In an alternate method provided for herein, complete hydatidiform mole may be differentially diagnosed in a subject by detecting the complexes formed between an anti-IPL nucleic acid and a target nucleic acid in a sample of suspected molar tissue from the subject. Preferably, the anti-IPL nucleic acid would specifically bind or hybridize to a target nucleic acid encoding amino acid residues 102-120 of human IPL protein. In an alternate preferred embodiment, the anti-IPL nucleic acid would specifically hybridize to a target nucleic acid encoding amino acid residues 134-152 of human IPL protein.

In either of the foregoing methods, the detection of zero or near zero levels of complex formation in relation to a suitable control (such as levels of complex formation in normal placental tissue) indicates a diagnosis of complete hydatidiform mole, whereas the detection of normal or near normal levels of complex formation in relation to a suitable control would suggest a diagnosis of partial hydatidiform mole or hydropic abortion with molar characteristics.

Finally, the present invention discloses kits for use in the differential diagnosis of complete hydatidiform mole. In one embodiment, the kits comprise both (i) an anti-IPL antibody, or an antigen binding fragment thereof, which specifically binds to human IPL protein, and (ii) means for detecting the formation of complexes between the anti-IPL antibody or antigen binding fragment and the targeted protein. In an alternate embodiment, the kits comprise both (i) an anti-IPL nucleic acid which specifically binds to a target nucleic acid encoding human IPL protein, and (ii) means for detecting the formation of complexes between the anti-IPL nucleic acid and the target nucleic acid.

Additional aspects of the present invention will be apparent in view of the description that follows.

### BRIEF DESCRIPTION OF THE FIGURES

FIGS. 1A and 1B illustrate the expression of IPL mRNA in various human tissues. In FIG. 1A, Northern blots of poly-A+RNA from human organs were hybridized with a full-length IPL cDNA probe. IPL is selectively expressed in the placenta. Organs in the left panel were also examined in a previous study (Qian, et al., The IPL gene on chromosome 11p15.5 is imprinted in humans and mice and is similar to TDAG51, implicated in Fas expression and apoptosis. Hum Mol. Genet. 6:2021-2029, 1997). The right panel shows expression in additional organs. While there is minimal IPL mRNA in many organs, the placenta shows by far the strongest signal. The IPL blots were deliberately over-exposed to highlight the tissue-specificity of IPL expression. He, heart; Br, brain; FT-P1, full term placenta; Lu, lung; Li, liver; Mu, skeletal muscle; Ki, kidney; Pa, pancreas; St, stomach; Td, thyroid; SC, spinal cord; LN, lymph node; Tr, trachea; Ad, adrenal; BM, bone marrow. FIG. 1B shows Northern blots of total RNA from human placentas over a range of gestational ages, hybridized with the indicated probes (anti-IPL or monoclonal anti-beta-actin). Weeks of gestation are indicated above each lane, while “FT” denotes a series of full term placentas. IPL mRNA is easily detectable throughout gestation. Variation in expression among placentas at a given gestational stage may reflect variable amounts of free chorionic villi in the different samples. An additional comparison of IPL mRNA in full-term vs. early gestation placentas can be seen in the control lanes of FIG. 6B.

FIGS. 2A-2D show the immunohistochemical localization of IPL protein in human placentas at early and mid-gestation. In FIG. 2A, a section of placental villi at 5 weeks gestation was immunostained with anti-IPL. The strongest IPL immunostaining is in the continuous layer of villous cytotrophoblast (vct), while the syncitiotrophoblast (st) is negative for IPL. There is weaker staining of some mesenchymal cells in the villous cores. FIG. 2B is from a section of placenta at 13 weeks gestation, and shows a similar pattern of IPL protein expression. FIG. 2C shows placenta at 13 weeks. Weak expression of IPL can be seen in an intervillous trophoblast island (ivt). Finally, FIG. 2D shows weak expression of IPL in extravillous cytotrophoblast (cytotrophoblast columns, cc) in a placenta at 13 weeks gestation. The C102 anti-IPL antibody was used in FIGS. 2A-C, and the C134 anti-IPL antibody was used in FIG. 2D.

FIGS. 3A and 3B illustrate the subcellular localization of IPL during human placental development. FIG. 3A shows a high-power (40×) image of IPL in the cytotrophoblast (vct) at 5 weeks gestation. The IPL protein is predominantly cytoplasmic. The syncitiotrophoblast (st) is negative for IPL. In contrast, as shown in FIG. 3B, by 25 weeks the placenta IPL has become predominantly nuclear, with reduced but still positive cytoplasmic staining. The majority of cytotrophoblast cells remain IPL-positive. Both sections were immunostained with the C102 anti-IPL antibody.

FIGS. 4A-4D show the co-expression of IPL and p57_{KIP2} in villous cytotrophoblast but not in other cell types. FIG. 4A is a section of 13 week gestation placenta, developed with anti-IPL (C102 antibody). FIG. 4B is a serial section of the placenta, developed with anti-p57_{KIP2}. FIG. 4C illustrates 15 week gestation fetal intestine, developed with anti-IPL. No positive cells are present. In FIG. 4D, a serial section of the intestine developed with anti-p57_{KIP2} shows strong nuclear staining in numerous epithelial cells.

FIGS. 5A-5D show the lack of IPL protein expression in complete hydatidiform moles. FIG. 5A is a section of complete hydatidiform mole (25 week gestation twin pregnancy) developed with anti-IPL antibody. There is very faint or absent staining. A similar lack of immunoreactive IPL was found in a second case of complete hydatidiform mole (not shown). FIG. 5B is a section of normal 25 week placenta (twin of the hydatidiform mole in FIG. 5A) showing strong nuclear and cytoplasmic immunoreactivity for IPL in the discontinuous villous cytotrophoblast layer. FIG. 5C is a second case of complete mole immunostained with anti-IPL. Finally, FIG. 5D is a section of control full-term placenta, showing strong IPL staining in the discontinuous villous cytotrophoblast layer. The sections were developed with the C134 anti-IPL antibody.

FIGS. 6A and 6B illustrate the lack of IPL protein and IPL mRNA in complete hydatidiform moles, as shown by western and northern blotting. FIG. 6A is a Western blot analyzing expression of IPL protein. The IPL signal is a doublet centered at 18 kilodaltons (kd), which is absent in the complete hydatidiform mole (CHM). Gestational ages of the control placentas are indicated. In FIG. 6B, a Northern blot analysis shows that the IPL mRNA signal is absent or barely detectable in the complete moles, but is readily detected in the partial hydatidiform mole (PHM) and in the control placentas.

### DETAILED DESCRIPTION OF THE INVENTION

The inventors have established herein that IPL expression (i.e., IPL transcription or translation) may be used as a “negative marker” for the differential diagnosis of certain androgenetically derived trophoblastic diseases, such as complete hydatidiform mole. This utility as a “negative marker” stems from the fact that IPL is a paternally imprinted gene, and expression of IPL results almost entirely from transcription and translation of the maternal allele of the Ipl gene. Consequently, where the chromosomal complement of suspected molar tissue is paternally derived, as in the case of complete hydatidiform mole, there is an absence or near absence of IPL expression, relative to a suitable control. (i.e., levels of expression seen in normal placental tissue). Conversely, where the chromosomal complement of the suspected molar tissue is biparentally derived, as in the case of partial hydatidiform mole, or in cases of hydropic abortion with molar characteristics, levels of IPL expression are normal or nearly normal, relative to a suitable control.

Accordingly, the present invention describes various compositions comprising a binding agent, where the binding agent binds specifically to a human IPL target molecule. In one embodiment of the invention, the binding agent is an antibody or an antigen binding fragment of an antibody which binds specifically to an epitope of the human IPL protein. In another embodiment of the invention, the binding agent is a nucleic acid that binds or hybridizes specifically to a complementary nucleic acid encoding all or a portion of the human IPL protein.

In either embodiment, the binding of the binding agent to the target molecule is said to be “specific” where the binding agent selectively binds to its intended target molecule with minimum binding to unintended target molecules or background. While the binding agents of the present invention may be capable of binding unintended target molecules at a weak, but detectable level (e.g., 10% or less of the binding shown to the target molecule), such weak binding may be readily discernable from the specific binding to the target molecule by the use of appropriate controls.

Where the binding agent is an antibody or an antigen binding fragment of an antibody, the paratope of the antibody or antigen binding fragment will bind with high avidity and/or affinity to the human IPL protein, and preferably to a specific epitope of the human IPL protein, with minimum binding to non-target proteins or epitopes. Avidity is defined herein as the total combining power of an antibody with an antigen, and is therefore a measure of the overall stability of the antibody-antigen complex. Affinity refers to the innate binding strength of an antibody paratope with a single epitope.

Where the binding agent is a nucleic acid, binding is specific where the nucleic acid hybridizes to its intended target, and not to an unintended target, under stringent hybridization conditions. The conditions required for stringency will vary according to a number of factors, including nucleic acid concentration, ionic concentration of the hybridization buffer, the sequence composition of the probe, and temperature. Accordingly, there is no single set of high stringency conditions that will yield specific hybridization (i.e., level of base mismatch approaching zero between probe and target sequence) for every situation. Generally, as used herein, high stringency conditions are those conditions of nucleic acid concentration, ionic concentration and temperature yielding a stable duplex almost exclusively between anti-IPL nucleic acids and target nucleic acids having 0% nucleotide mismatch for a probe length of 25 mers and under, and a stable duplex or heteroduplex for anti-IPL nucleic acid lengths longer than 25 mers, wherein there is a 0% mismatch for any stable duplex, and less than a 5% mismatch for any stable heteroduplex. Stringency may be increased though the addition of denaturing agents such as formamide or urea which lower the melting temperature of a nucleic acid duplex or heteroduplex. Subject to the foregoing variables, high stringency conditions may be generally defined as including use of a prewashing solution of 5×SSC, 0.5% SDS, 1.0 mM EDTA (pH 8.0), followed by hybridization conditions of about 50% formamide, 6×SSC at about 42° C., and washing conditions of approximately 68° C., 0.2×SSC, and 1% SDS. (See, Sambrook et al., Molecular Cloning: A Laboratory Manual, 2ed. Vol. 1, pp. 1.101-104, Cold Spring Harbor Laboratory Press, (1989)).

In one embodiment of the present invention, the binding agent of the composition is an anti-IPL antibody or an antigen binding fragment thereof, wherein the antibody or antibody fragment specifically binds to a human IPL protein. Preferably, the anti-IPL antibody or antigen binding fragment of the present invention is raised against and is targeted to a specific epitope of the human IPL protein. As used herein, “epitope” refers to the antigenic determinant on the IPL protein to which the antibody or antibody fragment of the present invention binds.

In one preferred embodiment of the invention, the target epitope of the human IPL protein comprises all or part of the sequence of amino acid residues QNRRALQDFRSRQERTAPA (SEQ ID NO.: 1), where SEQ ID NO.: 1 consists of residues 102-120 of the human IPL protein. (Qian, et al., supra, 1997). In an alternate preferred embodiment, the antibody or antibody fragment of the composition specifically binds to a target epitope comprising all or part of the sequence of amino acid residues PSEPSEPSRPSPQPKPPRTP (SEQ ID NO.: 2), where SEQ ID NO.: 2 consists of residues 134-152 of the human IPL protein. (Qian, et al., supra, 1997). Most preferably, the epitopes of the human IPL protein specifically binding to the antibodies or antibody fragments of the present invention consist of amino acid sequence QNRRALQDFRSRQERTAPA (SEQ ID NO.: 1), amino acid sequence PSEPSEPSRPSPQPKPPRTP (SEQ ID NO.: 2), or an antigenic portion of either amino acid sequence.

Methods of generating the antibodies or antibody fragments of the present invention are well known, and will be readily apparent to one of ordinary skill in the art. The antibodies of the composition may be single chain antibodies (see Ladner, et al., U.S. Pat. No. 4,946,778, entitled “Single polypeptide chain binding molecules”), monoclonal antibodies (see, E. Harlow and D. Lane, Eds., in “Antibodies—A Laboratory Manual”, Cold Spring Harbor Laboratory, 1996), polyclonal antibodies (Harlow and Lane, supra, 1996), humanized antibodies (see Gregory Winter, U.S. Pat. No. 5,225,539, entitled “Recombinant altered antibodies and methods of making altered antibodies”), or chimeric antibodies (see Cabilly, et al., U.S. Pat. No. 4,816,567, entitled “Recombinant immunoglobin preparations”). Alternatively, they may be the antigen binding fragments of any of the foregoing, including, but not limited to, a Fab, F(ab ¹)₂ or Fv fragment. These fragments may be generated by conventional recombination DNA techniques (Huse, W. D., et al., Generation of a large combinatorial library of the immunoglobulin repertoire in phage lambda. Science. 246:1275-1281, 1989) or by digestion of the antibody molecule with proteolytic enzymes, such as pepsin or papain.

Most commonly, antibodies are raised by the repeated immunization of a host animal, such as a donkey, horse, rat, mouse, goat, or preferably, a rabbit, with a suspension comprising the human IPL antigen. In a preferred (and exemplified) method, the antigen is an artificial peptide comprising a short (i.e., 6 to 20) sequence of amino acid residues from the human IPL protein, where the artificial peptide is coupled to an immunogenic carrier molecule via a free sulfhydryl containing cysteine residue (or other suitable reactive group). Common immunogenic carrier molecules include keyhole limpet hemocyanin, bovine serum albumin, ovalbumin and PPd, a hapten protein derivative of tuberculin. Preferably, the non-specific immune response of the host animal is further strengthened by the simultaneous injection of an adjuvant, such as Freund's (complete and/or incomplete), mineral gel, an oil emulsion, dinitrophenol, or a lecithin derivative. Monoclonal antibodies, expressing a single antibody directed to a single epitope, are most commonly generated by the fusion of lymphoid cells from the spleen of the immunized animal with immortal mycloma cell lines. The resulting hybridomas can then be plated and selected for secretion of the desired antibody.

The invention further provides a composition comprising an anti-IPL nucleic acid, wherein the anti-IPL nucleic acid specifically binds or hybridizes to a target nucleic acid encoding all or a portion of the human IPL protein. The anti-IPL nucleic acid may consist of the entire nucleotide coding sequence of the human IPL protein, but in a preferred embodiment of the invention, the anti-IPL nucleic acid specifically binds to a target nucleic acid encoding all or part of the amino acid sequence QNRRALQDFRSRQERTAPA (SEQ ID NO.: 1), which comprises residues 102-120 of the human IPL protein. In an alternate preferred embodiment, the anti-IPL nucleic acid specifically binds to a target nucleic acid encoding all or part of the amino acid sequence PSEPSEPSRPSPQPKPPRTP (SEQ ID NO.: 2), which comprises residues 134-152 of the human IPL protein. The anti-IPL nucleic acid may be any nucleic acid that binds to a complementary target mRNA sequence associated with the expression of the human Ipl gene. Accordingly, the nucleic acid may be any one of a double stranded RNA, single stranded RNA or cDNA molecule.

In a preferred embodiment of the invention, the composition further comprises a detectable label that would allow, directly (where the label is attached directly to the binding agent) or indirectly (i.e., via a labeled secondary binding agent), for the visualization or detection of complexes formed between the desired binding agent and its associated IPL target using standard immunoassay or imaging techniques. A large number of suitable detectable labels are well known in the art, including, but not limited to: enzymatic labels (malate dehydrogenase, horseradish peroxidase, biotin/avidin/streptavidin complex, alkaline phosphatase, beta-galactosidase, yeast alcohol dehydrogenase, alpha-glycerophosphate, triose phosphate isomerase, aspariginase, glucose oxidase, ribonuclease, urease, catalase, glucose-6-phosphate dehydrogenase, glucoamylase, etc.); fluorescent labels (green fluorescent protein, rhodamine, phycocyanin, allophycocyanin, phycoerythrin, o-phthaldehyde, fluorescein, fluorescein isothiocyanate, etc.); chemiluminescent labels (imidazole, acridinium salt, luminol, isoluminol, theromatic acridinum ester, oxalate ester, etc.); bioluminescent labels (luciferin, luciferase, aequorin, etc.); or radioactive labels (most commonly, ³H, ¹³¹I and ⁹⁹Tc).

The present invention still further discloses various methods to differentially diagnose complete hydatidiform mole in subject, wherein the sampled tissue is suspected to be one of complete hydatidiform mole, partial hydatidiform mole or hydropic abortion. The method comprises the steps of contacting a sample of suspected molar tissue from the subject with an anti-IPL antibody, or an antigen binding fragment thereof, and thereafter detecting the bound complexes formed between the anti-IPL antibody or antigen binding fragment and human IPL in the sample. The detection of zero or near zero levels of complex formation in relation to a suitable control indicates a diagnosis of complete hydatidiform mole, where a suitable control might be the level of complex formation seen in a sample of normal (i.e., biparental) placental tissue. As used herein, an absence or near absence of IPL expression is defined as a level that is less than 5% of the level of expression seen in normal placental tissue.

In a preferred method of the invention, the anti-IPL antibody or antigen binding fragment thereof binds specifically to an epitope of the human IPL protein comprising all or part of the amino acid sequence QNRRALQDFRSRQERTAPA (SEQ ID NO.: 1), where SEQ ID NO.: 1 consists of residues 102-120 of the human IPL protein. In an alternate preferred method of the invention, the anti-IPL antibody or antibody fragment specifically binds to a target epitope comprising all or part of the amino acid sequence PSEPSEPSRPSPQPKPPRTP (SEQ ID NO.: 2), where SEQ ID NO.: 2 consists of residues 134-152 of the human IPL protein. Most preferably, the anti-IPL antibody or antigen binding fragment specifically binds to the human IPL epitopes consisting of amino acid sequence QNRRALQDFRSRQERTAPA (SEQ ID NO.: 1) or amino acid sequence PSEPSEPSRPSPQPKPPRTP (SEQ ID NO.: 2), or an antigenic portion of either amino acid sequence. As noted above, the antibodies used in the method of the present invention may be monoclonal, polyclonal, humanized, chimeric, or single chained. Where an antigen binding fragment is used, the fragment may be any fragment (i.e., a Fab, F(ab¹)₂ or Fv fragment) where the antigen binding site specifically recognizes human IPL protein or a specific epitope of human IPL protein.

Methods and assays for detecting and quantifying the bound complexes formed between the anti-IPL antibody or antigen binding fragment and human IPL in the sample are well known in the art, and may include, but are not limited to, western blotting, enzyme linked immunosorbent assays, and immunohistochemical analysis. Western blotting is one of the oldest and most widely used methods to determine the presence and quantity of an antigen in a sample. Using this method, a sample of the suspected molar tissue is lyzed and prepared in an electrophoresis buffer. The proteins within the tissue suspension are separated using gel electrophoresis according to molecular weight. The proteins are then transferred to a solid support, such as a nitrocellulose membrane, via capillary blotting or electroblotting. Non-specific binding on the membrane can be blocked by incubation in a protein solution containing 10% (w/v) BSA or 5% non fat dried milk in phosphate buffer solution. Following a decanting step, a diluted solution of the antibody may be added, and allowed to incubate for an appropriate period of time (i.e., 30 minutes at 37° C., 1 hour at 25° C., or overnight at 4° C.) to allow for formation of the antibody-antigen complex. Formation of the antibody-antigen complexes is most commonly detected through use of an enzymatic label (i.e., alkaline phosphatase or horseradish peroxidase, either attached directly to the primary antibody, or to a secondary antibody targeted to recognize the primary antibody) that converts a substrate to a colored precipitate at the site of antibody binding, although any suitable label may be used (i.e., chemiluminescent labels, fluorescent labels, bioluminescent labels or radioactive labels).

Formation of antibody-antigen complexes may also be detected and quantified using an enzyme linked immunosorbent assay, or ELISA. In this method, two antibodies or antibody fragments are required that have distinct and unique epitopes on the human ILP protein. Accordingly, in a preferred ELISA of the present invention, one antibody (or antibody fragment) would bind specifically to all or part of amino acid residues QNRRALQDFRSRQERTAPA (SEQ ID NO.: 1), while another antibody (or antibody fragment) would bind specifically to all or part of amino acid residues PSEPSEPSRPSPQPKPPRTP (SEQ ID NO.: 2). The capture antibody is purified and bonded to a solid phase support, such as the bottom of a 96 well plate. It is then brought into contact with the antigen (derived from suspected molar tissue), where the capture antibody-antigen complex is allowed to form. Following the removal of all unbound products, the second antibody is contacted with the bound antigen, allowing for the formation of a “sandwich” comprising the first bound antibody, the desired target antigen, and the second bound antibody. An enzymatic label attached to the second antibody allows for the detection of bound antigen-antibody complexes by converting a clear substrate to a colored product. By way of example, p-nitrophenylphosphate can be converted to yellow p-nitrophenol by the using alkaline phosphatase as a label. Further, a peroxidase label can be used with ABTS (2,2′-azo-bis(3-ethylbenzthiazoline-6-sulfonic acid), OPD (o-phenylenediamine) or TMB (3,3′,5,5′-tetramethylbenzidine base) substrate to yield a colored product. The concentration of bound antigen can be determined colorimetrically using the appropriate spectroscopy equipment.

In a most preferred method of the present invention, the bound IPL protein would be detected using immunohistochemical methods, since these methods would allow for the visualization of IPL expression in situ within intact cells and according to cell type. Using immunohistochemical methods, the anti-IPL antibody is used to link the IPL antigen with a stain that can be visualized using light microscopy. Sections of the suspected molar tissue must first be treated with a fixative to preserve the sampled cells. The choice of fixative will depend largely upon what type of section is used (cryostat or paraffin embedded), the thickness of the tissue, as well as cell type sampled. Generally, aldehydes, such as formalin, formaldehyde, glutaraldehyde, or paraformaldehyde, are used, although acetone and methanol are sometimes used to fix proteins by denaturation. Optimal fixatives and fixation times will vary in each situation, but will be readily apparent to one of ordinary skill in the art. An antigen retrieval step follows fixation, where the availability of the IPL antigen to the anti-IPL antibody or antibody fragment is maximized. Antigen retrieval may involve enzymatic digestion, or autoclave or pressure cooking, but most preferably involves microwave irradiation in a suitable buffer solution (i.e., irradiation for 8 minutes at maximum power, in 0.01 M citrate, pH 6.0; 0.1 M Tris HCl, pH 8.0; 1 mM EDTA, pH 8.0). Following fixation, the section is immersed in an anti-IPL antibody dilution, which is either labeled directly or comprises a secondary labeled antibody. Detection may be by any known method, but is most commonly enzyme mediated (e.g., horseradish peroxidase, alkaline phosphatase), fluorophore mediated (e.g., fluorescein, rhodamine, Texas Red, Cy3, Cy5) or mediated using colloidal gold.

The present invention still further provides a method for differentially diagnosing complete hydatidiform mole in a subject, comprising the steps of contacting a sample of suspected molar tissue with an anti-IPL nucleic acid, and therafter detecting complexes formed between the anti-IPL nucleic acid and a target nucleic acid encoding human IPL in the sample, preferably by Northern blotting or immunohistochemical techniques. The detection of zero or near zero levels of complex formation in relation to a suitable control indicates a diagnosis of complete hydatidiform mole. The anti-IPL nucleic acid can be any one of a number of nucleic acid molecules that binds to a mRNA of complementary nucleic acid sequence, such as a double stranded RNA, single stranded RNA or a cDNA. While the target nucleic acid may be the entire coding sequence of the IPL protein, in a preferred embodiment of the invention, the anti-IPL nucleic acid specifically hybridizes to a target nucleic acid encoding all or a portion of amino acid residues 102-120 (SEQ ID NO.: 1) of human IPL protein. In another preferred embodiment of the invention, the anti-IPL nucleic acid specifically hybridizes to a target nucleic acid encoding all or portion of amino acid residues 134-152 (SEQ ID NO.: 2) of human IPL protein.

The invention further provides kits for use in the differential diagnosis of complete hydatidiform mole, comprising an anti-IPL antibody or antigen binding fragment thereof which specifically binds to amino acid residues 102-120 (SEQ ID NO.: 1) of the human IPL protein, together with means for detecting the formation of complexes between the anti-IPL antibody or antigen binding fragment and the targeted amino acid residues. In another kit of the present invention, the provided anti-IPL antibody or antigen binding fragment thereof specifically binds to amino acid residues 134-152 (SEQ ID NO.: 2) of the human IPL protein.

The invention further provides for a kit for use in the differential diagnosis of complete hydatidiform mole, comprising an anti-IPL nucleic acid which specifically binds to a target nucleic acid encoding amino acid residues 102-120 (SEQ ID NO.: 1) of the human IPL protein, together with means for detecting the formation of complexes between the anti-IPL nucleic acid and the targeted nucleic acid. In another kit of the present invention, the provided anti-IPL nucleic acid specifically binds to a target nucleic acid encoding amino acid residues 134-152 (SEQ ID NO.: 2) of the human IPL protein.

The present invention is described in the following Examples, which are set forth to aid in the understanding of the invention, and should not be construed to limit in any way the scope of the invention as defined in the claims that follow thereafter.

### EXAMPLES

### Example 1

### Generation of Anti-IPL Antibodies

Two C-terminal human IPL peptide sequences were custom-synthesized using traditional carrier carrier protein (KLH) conjugation via an artificial cysteine residue at the amino-terminus (Invitrogen Corporation, Carlsbad, Calif.). They were designated as hIPL-C102 (CQNRRALQDFRSRQERTAPA) (SEQ ID NO.: 3) and hIPL-C134 (CPSEPSEPSRPSPQPKPRTP) (SEQ ID NO.: 4), respectively. The resin-cleaved grade synthesized peptides were then injected into New Zealand white rabbits (range 3-9 months of age) to generate specific polyclonal anti-peptide antibodies. A standard protocol of twelve to sixteen weeks was used for immunization and obtaining antiserum. Specifically, KLH-peptide was emulsified by mixing with an equal volume of Freund's Adjuvant, and injected into three subcutaneous dorsal sites, for a total of 0.1 mg peptide per immunization. Animals were bled from the auricular artery. The blood was allowed to clot and the serum was then collected by centrifugation. The anti-peptide antibody titer was determined with an ELISA (enzyme linked immunosorbent assay) with free peptide bound in solid phase (μg/well). The antibody titer was subsequently confirmed with western blot analysis. The antibody representing the best titer in ELISA and western blot analysis was used further for affinity-purification.

The polyclonal anti-peptide antibodies against hIPL were affinity purified. The sulfhydryl group in the peptide was covalently coupled to the immobilized iodoacetyl on cross-linked agarose beads using the SulfoLink affinity purification column (Pierce Biotechnology, Inc., Rockford, Ill.). 1 mg of sulfhydryl containing peptide per ml of coupling gel was used. The standard protocol supplied by the manufacturer was followed.

Antiserum presenting high titer antibodies was then diluted with phosphate buffered saline (PBS) to obtain a pH of 7.5. Approximately 7 ml of original serum was applied to 1 ml of peptide-agarose column. The standard protocol provided by manufacturer was used for the sample application, washing the affinity column and elution of purified antibodies. Specific antibodies against hIPL-peptide were eluted in glycine buffer (100 mM, pH 2.5-3.0) and subsequently neutralized by 1M Tris, pH9.5. The antibody typically eluted out in 2^{nd} and 3^{rd} fractions (bed volume of the column) at low pH. The fractions of interest were pooled and dialyzed against 20 mM Tris, pH 8.0, 100 mM NaCl, 0.2mM EDTA and 20% glycerol. The aliquots of the purified antibodies were then stored at −70° C. The purified antibodies were used at 1:5000 (C102) or 1:6000 (C134) dilution.

### Example 2

### Immunohistochemistry

Tissue sections were deparaffinized in xylene and hydrated through graded ethanols. Antigen retrieval was carried out in 1 mM EDTA buffer by boiling the slides in a microwave oven for 8 minutes at the maximum power. Endogenous biotin was blocked by two incubations for 10 minutes with egg white and then with 5% fat-free milk in 1× Tris-buffered saline containing 0.01 % Tween 20 (TBST). Between the two blocking steps for biotin, slides were treated with 0.3% hydrogen peroxide in distilled water to block endogenous peroxidase activity. Slides were washed three times in 1× TBST and then incubated with 5% normal goat serum in 1× TBST containing 0.5% BSA for 30 minutes in a humidified chamber. After three TBST washes, slides were incubated with affinity-purified polyclonal antipeptide anti-hIPL antibodies or with a polyclonal anti-peptide antiserum against p57_{KIP2} (C-20; Santa Cruz Biotechnology, Santa Cruz, Calif.; utilized at 1:1000) at room temperature overnight. As described in Example 1, two peptide antibodies against synthetic peptides were generated, using a KLH carrier protein to create the immunogen. Following the primary antibody incubation the slides were washed three times in TBS-T and subsequently incubated with biotinylated secondary antibodies (Vector Laboratory, Burlingame, Calif.) for 30-40 minutes at room temperature. Antigen-antibody complexes were developed using Vectastain ABC kits (Vector Laboratory, Burlingame, Calif.) and a chromogenic substrate, diaminobezidine (DakoCytomation, Carpinteria, Calif.). Sections were counterstained with hematoxylin. As a control, pre-blocking of the antiserum was carried out with an excess of the cognate peptides, and this eliminated the staining. The two antibodies gave similar results on all tissues in repeated experiments, but with less stromal background staining when using C134.

### Example 3

### Northern Blotting

For northern analysis, total RNA from placentas and the hydatidiform mole was prepared using Trizol reagent (Invitrogen Corporation, Carlsbad, Calif.), and was then resolved on formaldehyde-containing agarose gels and transferred to Nytran membranes (Schleicher and Schull, Keene, N.H.). Hybridization with IPL and beta-actin cDNA probes was in ULTRAhyb buffer (Ambion, Inc., Austin, Tex.) at 42° C. Probes were stripped between hybridizations by boiling the membranes in 0.1 % SDS/0.1×SSC solution for 2 minutes. Frozen placental tissue samples were crushed under liquid N₂ and immediately boiled for 10 min in 2×SDS-PAGE loading buffer (100 mM Tris pH 6.8, 4% SDS, 0.2% bromophenol blue, 20% glycerol and 5% (v/v) 2-mercaptoethanol). Approximately 30 μg of total protein from placental samples was subjected to 12% Tris-glycine SDS-polyacrylamide gels (Invitrogen Corporation, Carlsbad, Calif.).

### Example 4

### Western Blotting

Proteins were transferred to nitrocellulose membranes. Membranes were blocked with 5% fat-free milk in 1×PBST (phosphate buffered saline containing 0.1% Tween20) and subsequently incubated with anti-IPL (1:3000) at 4° C. overnight or monoclonal anti-beta-actin (A5441, Sigma-Aldrich, Co., St. Louis, Mo.; utilized at 1:5000) at room temperature for 1 hr in 3% fat-free milk in 1×PBST. After washing, the membranes were incubated with goat peroxidase-conjugated anti-rabbit or anti-mouse IgG (Amersham Pharmarcia Biotech, Piscataway, N.J.) and the signal was detected using a commercial ECL detection system (Amersham Pharmarcia Biotech, Piscataway, N.J.).

### Example 5

### IPL mRNA is Expressed in the Human Placenta Throughout Gestation

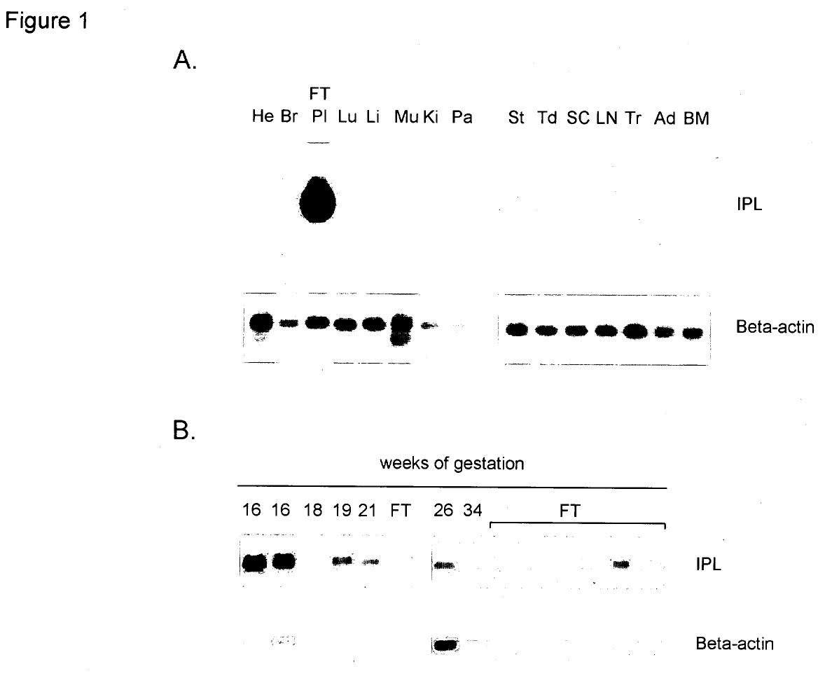

mRNA from multiple human tissues, including term placenta, was hybridized with a cDNA probe for human IPL (FIG. 1A). The resulting Northern blots confirmed that IPL expression is tissue specific, with the placenta being the only organ with very high levels of this transcript. This extends previous findings (Qian et al., The IPL gene on chromosome 11p15.5 is imprinted in humans and mice and is similar to TDAG51, implicated in Fas expression and apoptosis. Hum. Mol. Genet. 6:2021-2029, 1997) and parallels the tissue specificity of Ipl gene expression in the mouse (Qian, et al., supra, 1997; Frank et al., A novel pleckstrin homology-related gene family defined by Ipl/Tssc3, TDAG51, and Tih1: tissue-specific expression, chromosomal location, and parental imprinting. Mamm. Genome. 10:1150-1159, 1999). However, the high expression of human IPL in the placenta at term indicated a difference with the expression pattern of this gene in the mouse. The inventors have previously shown that the amounts of Ipl mRNA in the murine placenta decline precipitously between 12.5 and 14.5 days post-coitum (dpc), reflecting the rapid disappearance of a specific Ipl-expressing cell population (the Type II trophoblast) in the labyrinthine layer over this time interval (Frank, et al., Placental overgrowth in mice lacking the imprinted gene Ipl. Proc. Natl Acad. Sci. U.S.A. 99:7490-7495, 2002). In contrast, substantial IPL mRNA persists throughout gestation in the human placenta, as indicated by northern blotting of samples from a range of gestational ages (FIG. 1B).

### Example 6

### IPL Protein Marks the Villous Cytotrophoblast and is Expressed Throughout Human Gestation

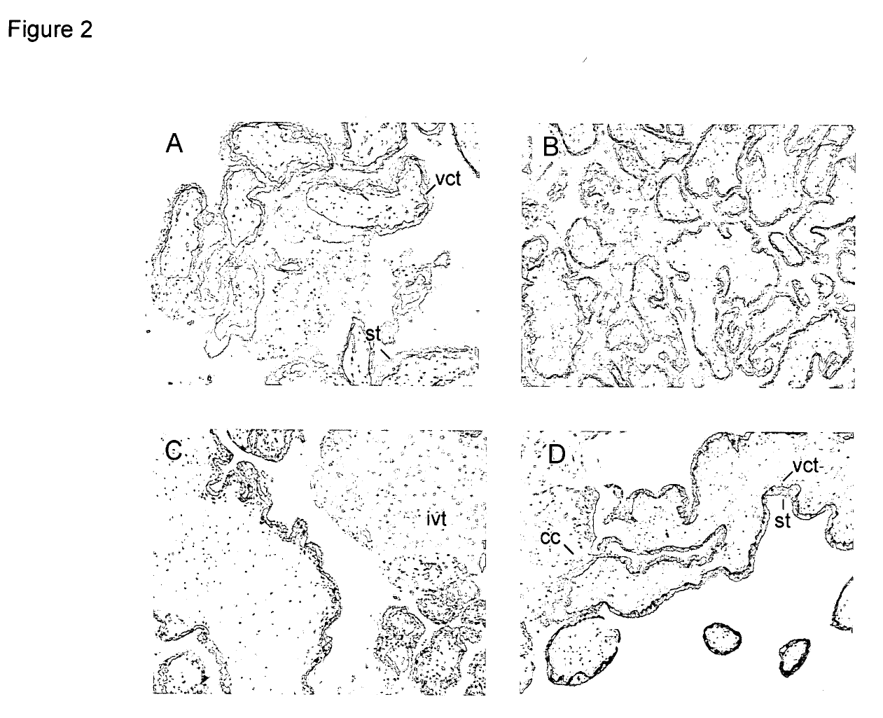

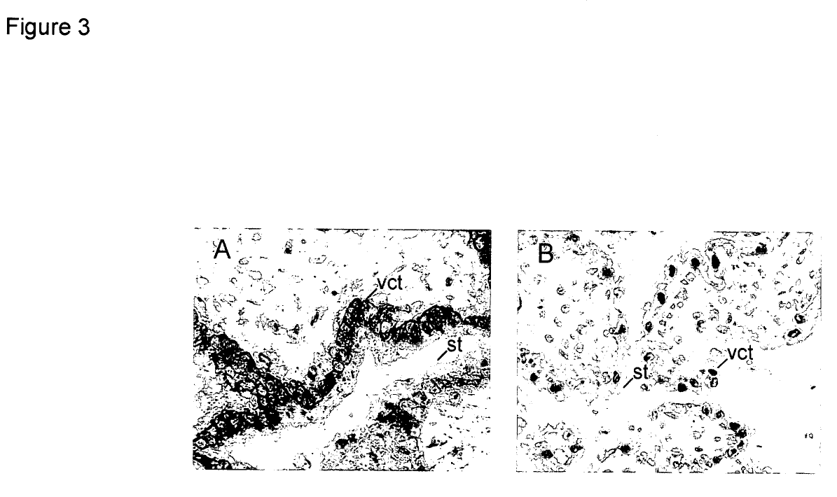

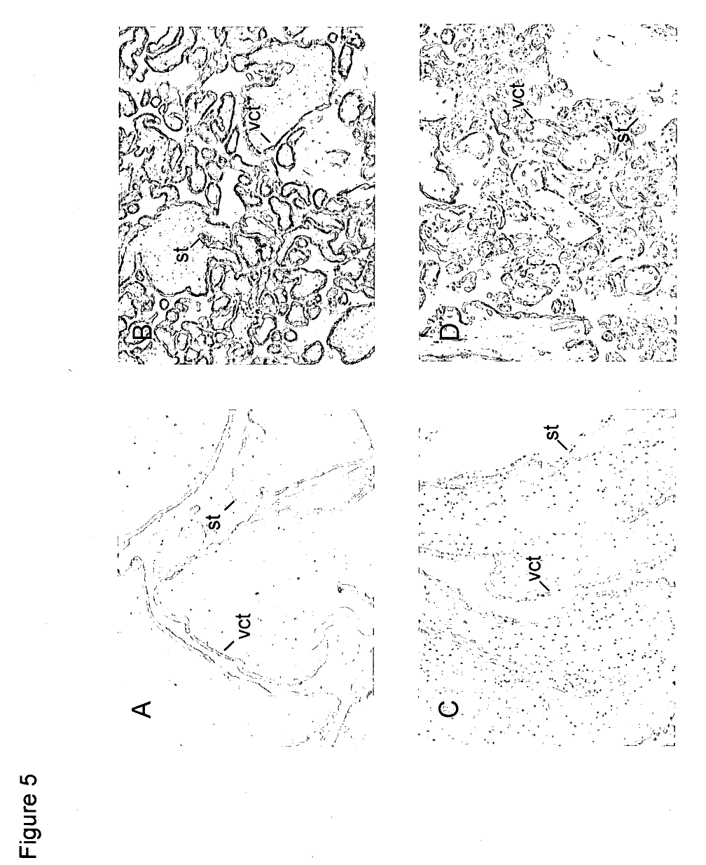

Two independent polyclonal anti-IPL antisera, raised against two different IPL peptide sequences (see Examples 1 and 2), were used for immunohistochemical analysis of sections of human placentas from a range of gestational ages. The results, shown in FIGS. 2-5, demonstrate strong expression of IPL protein in villous cytotrophoblast at all stages of gestation from the early chorionic sac to mature placentas at term. FIG. 2A shows IPL in villi from a chorionic sac corresponding to the 5ₜₕ postmenstrual week (<3 mm Crown Rump Length, Carnegie stage 10). At this early stage there is strong expression of IPL in the cytotrophoblast of tertiary villi throughout the chorionic sac. Syncytiotrophoblast cells are IPL-negative. Strong expression in the villous cytotrophoblast continues from this stage through the second and third trimesters, and persists until term (13 week placenta shown in FIGS. 2B-2D and term placenta shown in FIGS. 3B and 5D). As the cytotrophoblast becomes more dispersed with advancing maturation, the layer of immunopositive cells becomes correspondingly discontinuous. However, most or all of the cytotrophoblast cells remain IPL-positive, and the average intensity of staining in a given positive cell stays approximately the same during all stages of gestation. This “all or none” staining in the villous cytotrophoblast is analogous to the situation in mice, in which, despite the rapid loss of Ipl-positive cells at mid- to late-gestation, the intensity of staining per cell remains constant (Frank, et al., supra, 2002).

Staining for IPL is primarily cytoplasmic at early stages of human placental development (5 weeks gestation; FIG. 3A). However, at later stages, and definitely by 20 weeks of gestation (2.5 cm foot length), immunoreactive IPL becomes more strongly nuclear (FIG. 3B), with some persistent staining of the cytoplasm. The significance of this nuclear accumulation is not known, but it contrasts to the situation in the mouse placenta, in which the protein encoded by the Ipl gene remains predominantly, though not exclusively, cytoplasmic throughout development (Frank, et al., supra, 2002; Frank, et al., supra, 1999).

### Example 7

### IPL is Weakly Expressed in Extravillous Trophoblast

In addition to villous cytotrophoblast, IPL protein is detected at comparable levels in the extravillous cytotrophoblast of the chorionic plate (data not shown). However, extravillous trophoblast in the intervillous trophoblast islands (IVT) was only weakly positive for IPL (FIG. 2C). Similarly, IPL protein is present at reduced levels in the extravillous trophoblast of the basal plate. FIG. 2D shows anchoring villi with the usual intense staining of the villous cytotrophoblast, but with a substantial decline in IPL staining in cells of the extravillous trophoblast cell columns invading into the deeper layers of the basal plate.

### Example 8

### IPL and p57_{KIP2} are Co-Expressed in Villous Cytotrophoblast But Not in Other Cell Types

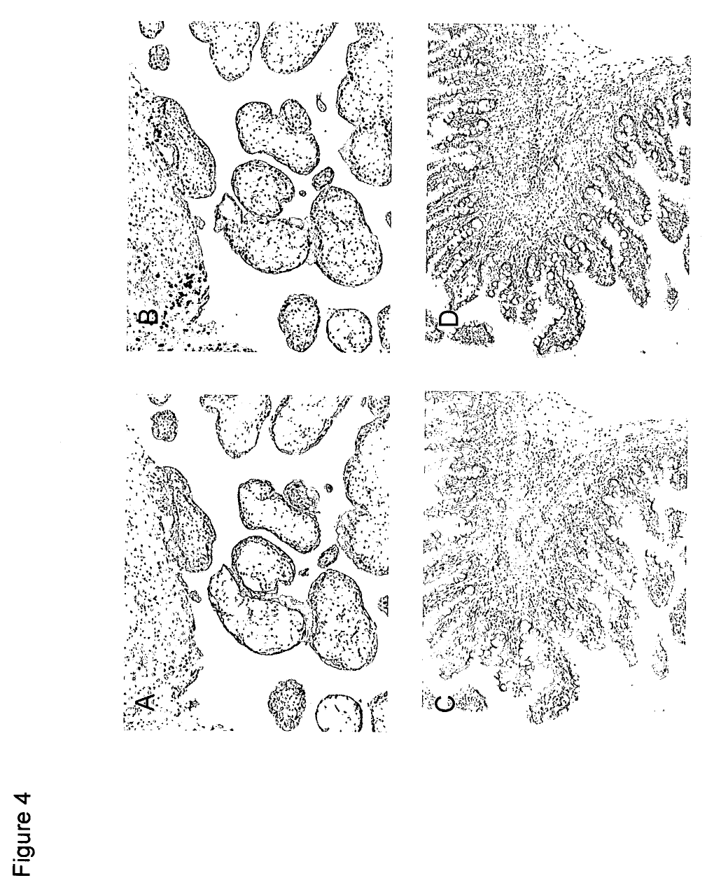

Data from KO mice have shown that the functional imprinting of Ipl/Tssc3 and p57_{Kip2}/Cdkn1c is controlled in cis by a single differentially methylated CpG-rich DNA sequence (imprinting center), the KvDMR1 element (Fitzpatrick et al., Regional loss of imprinting and growth deficiency in mice with a targeted deletion of KvDMR1. Nat Genet. 32:426-431, 2002). To determine whether these two genes are coordinately expressed in human placentas, serial sections of placentas were stained, as well as other fetal organs, with anti-IPL and anti-p57_{KIP2} antisera. Consistent with previous reports (Chilosi et al., Differential expression of p57kip2, a maternally imprinted cdk inhibitor, in normal human placenta and gestational trophoblastic disease. Lab. Invest. 78:269-276, 1998; Castrillon et al., Discrimination of complete hydatidiform mole from its mimics by immunohistochemistry of the paternally imprinted gene product p57KIP2. Am. J. Surg. Pathol. 25:1225-1230, 2001; Fukunaga, Immunohistochemical characterization of p57(KIP2) expression in early hydatidiform moles. Hum. Pathol. 33:1188-1192, 2002; Fisher et al., The maternally transcribed gene p57(KIP2) (CDNK1C) is abnormally expressed in both androgenetic and biparental complete hydatidiform moles. Hum. Mol. Genet. 11:3267-3272, 2002), p57_{KIP2} was seen in villous cytotrophoblast, the same tissue compartment that expresses IPL (FIGS. 4A and 4B). However, the cell-type distribution of p57_{KIP2} was considerably broader than that of IPL. Strong nuclear staining for p57_{KIP2} was observed not only in the villous cytotrophoblast, but also in the invasive extravillous cytotrophoblast. In addition, in a limited range of non-placental tissues examined, including intestinal epithelium, renal glomerular and tubular epithelium and lung epithelium, nuclear p57_{KIP2} was strongly expressed in the absence of IPL (see FIGS. 4C, 4D). Thus, IPL and p57_{KIP2}/CDKN1C, two closely linked and paternally imprinted genes, are coordinately expressed in villous cytotrophoblast, but as yet unidentified tissue-specific enhancers evidently influence the expression of p57_{KIP2} independently from IPL in extravillous trophoblast and in other human tissues.

### Example 9

### IPL Protein is Absent in Complete Hydatidiform Moles

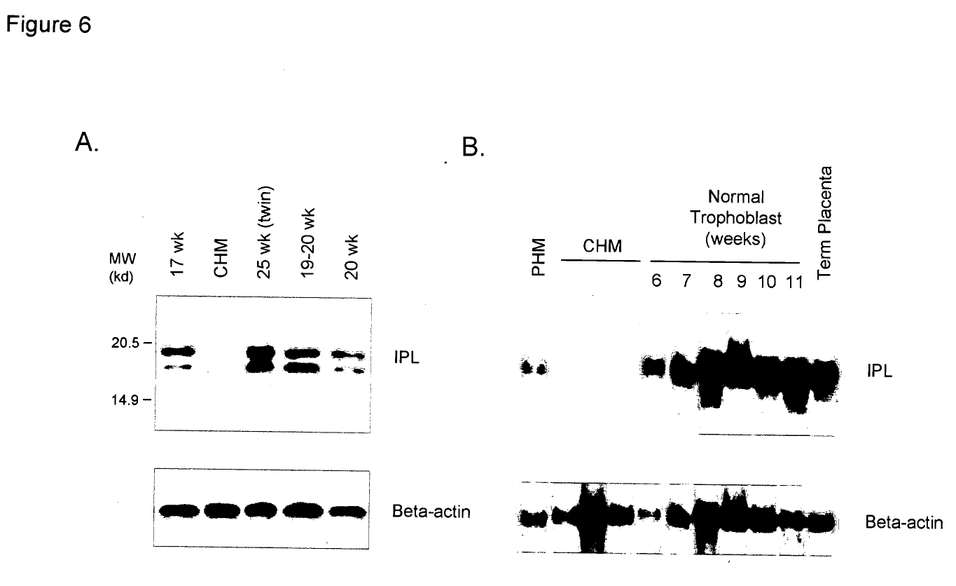

Given that human IPL is normally expressed predominantly from the maternal allele (Qian, et al., supra, 1997; Lee and Feinberg, Genomic imprinting of a human apoptosis gene homologue, TSSC3. Cancer Res. 58:1052-1056, 1998), it was reasoned that the net expression of this gene should be reduced in cells of complete hydatidiform moles, which are known to contain only paternally derived chromosomes. To examine this question, a case of a twin gestation was selected in which a complete hydatidiform mole co-existed with a stillborn twin male fetus attached to a non-molar placenta. Such cases, though rare, have been well described (Steller et al., Natural history of twin pregnancy with complete hydatidiform mole and coexisting fetus. Obstet. Gynecol. 83:35-42, 1994; Steller et al., Clinical features of multiple conception with partial or complete molar pregnancy and coexisting fetuses. J. Reprod. Med. 39:147-154, 1994). In this case, the two placentas were delivered at 25 weeks' gestation as separate specimens, not physically connected to each other. The non-molar placenta was 171 gm in weight, normal in gross and microscopic appearance, and associated with a morphologically normal male fetus. The molar placenta was a bulky specimen weighing 620 gm, consisting entirely of translucent cystic villi measuring up to 3.5 cm in diameter. Histology showed unequivocal features of a complete mole. Karyotypic analysis showed that the mole was 46, XX while the twin fetus and its non-molar placenta were 46, XY. Staining of sections of this complete hydatidiform mole showed virtual absence of IPL signal which, in contrast, was strong in the normal twin placenta of this same pregnancy (FIGS. 5A and 5B). The two different antibodies, anti-hIPL-C101L and C134, gave very similar results (data not shown). This result was verified by western blotting of the proteins extracted from the mole and its twin placenta. Human IPL protein is visualized as a closely spaced doublet, presumably due to a post-translational modification of a proportion of the protein, in western blotting of total protein lysates from normal placentas. Both bands derive from IPL, as shown by two criteria: first, the doublet is eliminated from IPL-expressing cell line lysates by RNA-interference, i.e. transfection of the cells with a double-stranded RNA oligonucleotide matching IPL mRNA sequences (A.S. and B.T., unpublished data); second, the identical doublet is seen with the two different anti-IPL antisera, directed against distinct peptide epitopes in the protein. Analysis by western blotting showed a nearly complete loss of the IPL protein bands in the complete mole (very faint specific bands were seen only on prolonged exposure of the films), while bands of normal intensity were detected in the non-molar twin placenta (FIG. 6A).

A second independent case of complete mole was examined, which like the first case showed classical histological features. As in the first case, there was little if any positive immunostaining of the molar tissue by anti-IPL, while control term placenta immunostained in parallel showed prominent IPL staining in the discontinuous villous cytotrophoblast (FIGS. 5C and 5D). Further evidence for a general absence of IPL expression in complete hydatidiform moles was provided by northern blot analysis of three additional cases (FIG. 6B). In this analysis, a case of partial mole, which is an abnormal trophoblastic proliferation that while karyotypically abnormal retains a maternal genetic contribution, was positive for IPL mRNA (FIG. 6B).

Loss of expression of the imprinted gene p57_{KIP2}/CDKN1C is likely an important determinant of growth hydatidiform moles, since the product of this gene is a cyclin-cdk inhibitor. Since knockout experiments in mice show a growth-restraining function for the Ipl gene (Frank, et al., 2002), loss of IPL expression might also contribute to growth of these neoplasms. Future studies will need to address this possibility and, more generally, to investigate the role of altered expression of imprinted genes, including both IPL and p57_{KIP2}/CDKN1C, in placental overgrowth and placental growth deficiency in the general population.

### Example 10

### Comparative Anatomy of Placental Structures

Despite differences in histological details, the mouse and human placentas are organized according to similar principles. As discussed in a recent review (Georgiades et al., Comparative developmental anatomy of the murine and human definitive placentae. Placenta. 23:3-19, 2002), the structural homologies are best appreciated by considering a section perpendicular to the fetal surface of the placenta. Nearest that surface is the labyrinthine zone in mice, which corresponds to the layer referred to as the “fetal placenta” in humans. This region is a major site of nutrient exchange, in which zygote-derived structures, the chorionic villi in humans and the maze-like ramifications of the labyrinth in mice, are exposed to maternal blood. In this zone, the placentas of both species show a similar structural organization, with fetal blood vessels contained within mesenchymal cores that are lined by trophoblast, which in turn contacts the maternal blood. However, the detailed arrangement of the trophoblast differs between the two species. In the mouse, and in the related trichorial hamster placenta (Carpenter, Ultrastructural observations on the maturation of the placental labyrinth of the golden hamster (days 10 to 16 of gestation). Am. J. Anat. 143:315-347, 1975), there are three closely apposed layers of trophoblast, each with a characteristic ultrastructural appearance. Nearest the maternal blood are Type I trophoblast cells, which can be identified in light microscopy by their location and their large nuclei, and which do not express Ipl mRNA or protein (Frank, et al., supra, 2002). In location, these cells might be considered homologous to human syncytiotrophoblast, which, as shown here, is also negative for IPL. Deep to the Type I cells in the mouse labyrinth are the Type II trophoblast cells, followed by Type III trophoblast, which produce long cytoplasmic extensions that surround the fetal capillaries, from which they are separated by a thin basement membrane (Sapin et al., Defects of the chorioallantoic placenta in mouse RXRalpha null fetuses. Dev. Biol. 191:29-411997). Type II cells are strongly Ipl-positive, and according to this shared marker, and by location (one cell removed from the maternal blood), they are likely homologous to IPL-positive human villous cytotrophoblast. The Type III cells in the mouse, which are weakly Ipl-positive (Frank, et al., supra, 2002), have no apparent counterpart in the human placenta. The placental layer deep to the labyrinth in the mouse is the junctional zone, which consists largely of spongiotrophoblast and which invades the maternal decidua. This layer is very weakly Ipl-positive in the mouse, and the corresponding region and cell type in the human placenta, the basal plate and the extravillous cytotrophoblast, also shows markedly reduced, though not absent, IPL expression.

There are additional markers that might support homology between Type II trophoblast of the mouse labyrinth and human villous cytotrophoblast. Gcm1, a transcription factor essential for growth of the labyrinth in mice (Anson-Cartwright et al., The glial cells missing-1 protein is essential for branching morphogenesis in the chorioallantoic placenta. Nat. Genet. 25:311-314, 2000; Schreiber et al., Placental failure in mice lacking the mammalian homolog of glial cells missing GCMa. Mol. Cell Biol. 20:2466-2474, 2000), is promising in this regard. Murine Gcm1 is expressed in a subset of labyrinthine trophoblast cells, which are concentrated near the chorionic plate, and which appear to be Type II cells by virtue of their presence in tight clusters located between the blood vessels (Basyuk et al., Murine Gcm1 gene is expressed in a subset of placental trophoblast cells. Dev. Dyn. 214:303-311, 1999). This pattern is similar to the distribution of Ipl-positive cells (Frank, el al., supra, 2002). In addition, the number of Gcm1-positive cells in the mouse placenta declines rapidly around mid-gestation, again paralleling the findings for the Ipl-positive cell population. Consistent with homology between Type II labyrinthine trophoblast and villous cytotrophoblast, recent analyses showed the highest amounts of GCM1 in the villous cytotrophoblast of the human placenta (Nait-Oumesmar et al., Placental expression and chromosomal localization of the human Gcm 1 gene. J. Histochem. Cytochem. 48:915-922, 2000; Janatpour et al., A repertoire of differentially expressed transcription factors that offers insight into mechanisms of human cytotrophoblast differentiation. Dev. Genet. 25:146-157, 1999). Other markers for human villous cytotrophoblast, such as c-MET (Clark et al., Hepatocyte growth factor/scatter factor and its receptor c-met: localisation and expression in the human placenta throughout pregnancy. J. Endocrinol. 151:459-467, 1996; Kauma et al., The differential expression of hepatocyte growth factor and met in human placenta. J. Clin. Endocrinol. Metab. 82:949-954, 1997; Saito et al., Hepatocyte growth factor promotes the growth of cytotrophoblasts by the paracrine mechanism. J. Biochem. (Tokyo). 117:671-6761995), ERK1/2 (Kita et al., Expression and Activation of MAP Kinases, ERK1/2, in the Human Villous Trophoblasts. Placenta. 24:164-172, 2003), and the alpha-, beta- and gamma-catenins (Getsios et al., Regulation of beta-catenin mRNA and protein levels in human villous cytotrophoblasts undergoing aggregation and fusion in vitro: correlation with E-cadherin expression. J. Reprod. Fertil. 119:59-68, 2000; Getsios et al., alpha-, beta-, gamma-catenin, and p120(CTN) expression during the terminal differentiation and fusion of human mononucleate cytotrophoblasts in vitro and in vivo. Mol. Reprod. Dev. 59:168-177, 2001) have, to the knowledge of the inventors, not been examined at high resolution for their distribution in the mouse placenta, although the c-Met/HGF and MAP kinase pathways are essential for development of this placental layer (Uehara et al., Placental defect and embryonic lethality in mice lacking hepatocyte growth factor/scatter factor. Nature. 373:702-705, 1995; Schmidt et al., Scatter factor/hepatocyte growth factor is essential for liver development. Nature. 373:699-702, 1995; Sachs et al., Essential role of Gabi for signaling by the c-Met receptor in vivo. J. Cell. Biol. 150:1375-1384, 2000; Giroux et al., Embryonic death of Mek1-deficient mice reveals a role for this kinase in angiogenesis in the labyrinthine region of the placenta. Curr. Biol. 9:369-372, 1999; Itoh et al., Role of Gabl in heart, placenta, and skin development and growth factor- and cytokine-induced extracellular signal-regulated kinase mitogen-activated protein kinase activation. Mol. Cell. Biol. 20:3695-3704, 2000).

There is circumstantial evidence from histology, as well as functional evidence from studies of trophoblast cell differentiation in tissue culture, supporting a stem cell or “transit-amplifying cell” function for the villous cytotrophoblast in the human placenta. This cell type proliferates continuously during placental growth throughout human gestation, but can also differentiate into two post-mitotic cell types, either syncytiotrophoblast or invasive extravillous trophoblast, depending on positional and environmental cues, for example, (Caniggia et al., Activin is a local regulator of human cytotrophoblast cell differentiation. Endocrinology. 138:3976-3986, 1997; Morrish et al., Functional regulation of human trophoblast differentiation. J. Reprod. Immunol. 39:179-195, 1998; Genbacev et al., Human cytotrophoblast expression of the von Hippel-Lindau protein is downregulated during uterine invasion in situ and upregulated by hypoxia in vitro. Dev. Biol. 233:526-536, 2001). The Type II labyrinthine trophoblast in the mouse placenta may also serve this function. Many of these cells incorporate BrdU in early to mid-gestation (up to 12.5 days post coitum, dpc) (Frank, et al., supra, 2002), and they become less abundant by morphological criteria in mid- to late-gestation as the labyrinth matures (Carpenter, Ultrastructural observations on the maturation of the placental labyrinth of the golden hamster (days 10 to 16 of gestation). Am. J. Anat. 143:315-347, 1975). Moreover, the rapid disappearance of Ipl-positive and Gcm1-positive cells around14.5 dpc, when placental growth in the mouse slows and then ceases (Louvi et al., Growth-promoting interaction of IGF-II with the insulin receptor during mouse embryonic development. Dev. Biol. 189:33-48, 1997), is also circumstantial evidence for a stem cell or transit-amplifying (i.e. self-renewing but not totipotent) function.

All publications, patent applications and issued patents cited in this specification are herein incorporated by reference as if each individual publication, patent application or issued patent were specifically and individually indicated to be incorporated by reference. Further, the earlier incorporation by reference of any specific publication, patent application or issued patent shall not negate this paragraph. The citation of any publication, patent application or issued patent is for its disclosure prior to the filing date of the subject application and should not be construed as an admission that the present invention is not entitled to antedate such disclosure by virtue of prior invention.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A composition comprising an anti-IPL antibody or an antigen binding fragment thereof, wherein the antibody or antibody fragment specifically binds to all or a portion of amino acid residues 102-120 (SEQ ID NO.: 1) of human IPL protein.

2. The composition of claim 1, wherein the antibody is a monoclonal antibody.

3. The composition of claim 1, wherein the antibody is a polyclonal antibody.

4. The composition of claim 3, wherein the antibody is hIPL-C102.

5. The composition of claim 1, wherein the antibody is a humanized antibody.

6. The composition of claim 1, wherein the antibody is a chimeric antibody.

7. The composition of claim 1, wherein the antibody is a single chain antibody.

8. The composition of claim 1, where the antigen binding fragment is a Fab, F(ab¹)₂ or Fv fragment.

9. The composition of claim 1, further comprising a detectable label.

10. The composition of claim 9, wherein the detectable label is an enzymatic label, a fluorescent label, a chemiluminescent label, a bioluminescent label or a radioactive label.

11. A composition comprising an anti-IPL antibody or an antigen binding fragment thereof, wherein the antibody or antibody fragment specifically binds to all or a portion of amino acid residues 134-152 (SEQ ID NO.: 2) of human IPL protein.

12. The composition of claim 11, wherein the antibody is a monoclonal antibody.

13. The composition of claim 11, wherein the antibody is a polyclonal antibody.

14. The composition of claim 13, wherein the antibody is hIPL-C134.

15. The composition of claim 11, wherein the antibody is a humanized antibody.

16. The composition of claim 11, wherein the antibody is a chimeric antibody.

17. The composition of claim 11, wherein the antibody is a single chain antibody.

18. The composition of claim 11, where the antigen binding fragment is a Fab, F(ab¹)₂ or Fv fragment.

19. The composition of claim 11, further comprising a detectable label.

20. The composition of claim 19, wherein the detectable label is an enzymatic label, a fluorescent label, a chemiluminescent label, a bioluminescent label or a radioactive label.

21. A composition comprising an anti-IPL nucleic acid, wherein the anti-IPL nucleic acid specifically binds to a target nucleic acid encoding all or a portion of amino acid residues 102-120 (SEQ ID NO.: 1) of human IPL protein.

22. The composition of claim 21, wherein the anti-IPL nucleic acid is one of double stranded RNA, single stranded RNA or a cDNA.

23. The composition of claim 21, wherein the target nucleic acid is a mRNA.

24. The composition of claim 21, further comprising a detectable label.

25. The composition of claim 24, wherein the detectable label is an enzymatic label, a fluorescent label, a chemiluminescent label, a bioluminescent label or a radioactive label.

26. A composition comprising an anti-IPL nucleic acid, wherein the anti-IPL nucleic acid specifically binds to a target nucleic acid encoding all or a portion of amino acid residues 134-152 (SEQ ID NO.: 2) of human IPL protein.

27. The composition of claim 26, wherein the anti-IPL nucleic acid is one of double stranded RNA, single stranded RNA or a cDNA.

28. The composition of claim 26, wherein the target nucleic acid is a mRNA.

29. The composition of claim 26, further comprising a detectable label.

30. The composition of claim 29, wherein the detectable label is an enzymatic label, a fluorescent label, a chemiluminescent label, a bioluminescent label or a radioactive label.

31. A method for differentially diagnosing complete hydatidiform mole in a subject, comprising the steps of:
(a) contacting a sample of suspected molar tissue from the subject with an anti-IPL antibody, or an antigen binding fragment thereof; and
(b) detecting complexes formed between the anti-IPL antibody or antigen binding fragment and human IPL in the sample;
wherein the detection of zero or near zero levels of complex formation in relation to a suitable control indicates a diagnosis of complete hydatidiform mole.

32. The method of claim 31, wherein the anti-IPL antibody or antigen binding fragment thereof binds specifically to all or a portion of amino acid residues 102-120 (SEQ ID NO.: 1) of human IPL.

33. The method of claim 32, wherein the antibody is a monoclonal antibody.

34. The method of claim 32, wherein the antibody is a polyclonal antibody.

35. The method of claim 34, wherein the antibody is hIPL-C120.

36. The method of claim 32, wherein the antibody is a humanized antibody.

37. The method of claim 32, wherein the antibody is a chimeric antibody.

38. The method of claim 32, wherein the antibody is a single chain antibody.

39. The method of claim 32, where the antigen binding fragment is a Fab, F(ab¹)₂ or Fv fragment.

40. The method of claim 31, wherein the anti-IPL antibody or antigen binding fragment thereof binds specifically to all or a portion of amino acid residues 134-152 (SEQ ID NO.: 2) of human IPL.

41. The method of claim 40, wherein the antibody is a monoclonal antibody.

42. The method of claim 40, wherein the antibody is a polyclonal antibody.

43. The method of claim 42, wherein the antibody is hIPL-C134.

44. The method of claim 40, wherein the antibody is a humanized antibody.

45. The method of claim 40, wherein the antibody is a chimeric antibody.

46. The method of claim 40, wherein the antibody is a single chain antibody.

47. The method of claim 40, where the antigen binding fragment is a Fab, F(ab¹)₂ or Fv fragment.

48. A method for differentially diagnosing complete hydatidiform mole in a subject, comprising the steps of:
(a) contacting a sample of suspected molar tissue from the subject with an anti-IPL nucleic acid; and
(b) detecting complexes formed between the anti-IPL nucleic acid and a target nucleic acid encoding human IPL in the sample;
wherein the detection of zero or near zero levels of complex formation in relation to a suitable control indicates a diagnosis of complete hydatidiform mole.

49. The method of claim 48, wherein the anti-IPL nucleic acid specifically hybridizes to a target nucleic acid encoding all or a portion of amino acid residues 102-120 (SEQ ID NO.: 1) of human IPL protein.

50. The method of claim 49, wherein the anti-IPL nucleic acid is one of a double stranded RNA, single stranded RNA or a cDNA.

51. The method of claim 49, wherein the target nucleic acid is a mRNA.

52. The method of claim 48, wherein the anti-IPL nucleic acid specifically hybridizes to a target nucleic acid encoding all or a portion of amino acid residues 134-152 (SEQ ID NO.: 2) of human IPL protein.

53. The method of claim 52, wherein the anti-IPL nucleic acid is one of a double stranded RNA, single stranded RNA or a cDNA.

54. The method of claim 52, wherein the target nucleic acid is a mRNA.

55. A kit for use in the differential diagnosis of complete hydatidiform mole, comprising:
(a) an anti-IPL antibody or antigen binding fragment thereof which specifically binds to all or a portion of amino acid residues 102-120 (SEQ ID NO.: 1) of the human IPL protein; and
(b) means for detecting the formation of complexes between the anti-IPL antibody or antigen binding fragment thereof and the targeted amino acid residues.

56. A kit for use in the differential diagnosis of complete hydatidiform mole, comprising:
(a) an anti-IPL nucleic acid which specifically binds to a target nucleic acid encoding all or a portion of amino acid residues 102-120 (SEQ ID NO.: 1) of the human IPL protein; and
(b) means for detecting the formation of complexes between the anti-IPL nucleic acid and the targeted nucleic acid.

57. A kit for use in the differential diagnosis of complete hydatidiform mole, comprising:
(a) an anti-IPL antibody or antigen binding fragment thereof which specifically binds to all or a portion of amino acid residues 134-152 (SEQ ID NO.: 2) of the human IPL protein; and
(b) means for detecting the formation of complexes between the anti-IPL antibody or antigen binding fragment thereof and the targeted amino acid residues.

58. A kit for use in the differential diagnosis of complete hydatidiform mole, comprising:
(a) an anti-IPL nucleic acid which specifically binds to a target nucleic acid encoding all or a portion of amino acid residues 134-152 (SEQ ID NO.: 2) of the human IPL protein; and
(b) means for detecting the formation of complexes between the anti-IPL nucleic acid and the targeted nucleic acid.


SEQUENCE LISTING (4 sequences)

SEQ ID NO: 1 - protein, 19 aa, Homo sapiens
QNRRALQDFRSRQERTAPA

SEQ ID NO: 2 - protein, 19 aa, Homo sapiens
PSEPSEPSRPSPQPKPRTP

SEQ ID NO: 3 - protein, 20 aa, Artificial sequence
  MOD_RES (1)..(1): artificial sequence; addition of amino terminal cysteine for sulfhydryl mediated conjugation to keyhole limpet hemocyanin carrier molecule
CQNRRALQDFRSRQERTAPA

SEQ ID NO: 4 - protein, 20 aa, Artificial sequence
  MOD_RES (1)..(1): artificial sequence; addition of amino terminal cysteine for sulfydryl mediated conjugation to keyhole limpet hemocyanin carrier molecule
CPSEPSEPSRPSPQPKPRTP